(19) United States
(12) Patent Application Publication
(10) Pub. No.: US 2025/0064610 A1
(43) Pub. Date: Feb. 27, 2025

(54) STENT WITH ENHANCED SCAFFOLD

(71) Applicant: Boston Scientific Scimed, Inc., Maple Grove, MN (US)

(72) Inventors: James Hemerick, Brooklyn Park, MN (US); Paul F. Chouinard, Maple Grove, MN (US)

(73) Assignee: Boston Scientific Scimed, Inc., Maple Grove, MN (US)

(21) Appl. No.: 18/810,647

(22) Filed: Aug. 21, 2024

Related U.S. Application Data
(60) Provisional application No. 63/534,613, filed on Aug. 25, 2023.

(51) Int. Cl. A61F 2/91

(52) U.S. Cl. CPC A61F 2/91

(57) ABSTRACT

A medical stent includes an inner metallic tubular member including a plurality of interconnected struts defining the inner metallic tubular member, and an outer metallic tubular member circumferentially surrounding the inner tubular member. The outer metallic tubular member includes one or more filaments knitted together in a plurality of loops that together form the outer metallic tubular member. The outer metallic tubular member is secured relative to the inner metallic tubular member. The inner tubular member exerts a radially outward force on the outer tubular member in a radially expanded configuration.

### CROSS-REFERENCE TO RELATED APPLICATIONS

The present application claims the benefit of U.S. Provisional Patent Application Ser. No. 63/534,613, filed Aug. 25, 2023, the disclosure of which is incorporated herein by reference.

### TECHNICAL FIELD

The present disclosure relates generally to methods and apparatuses for various ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure. These devices are manufactured by any one of a variety of different manufacturing methods and may be used according to any one of a variety of methods for a variety of applications. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

The disclosure is directed to several alternative designs, materials and methods of manufacturing medical device structures and assemblies, and the use thereof.

One example is a medical stent including an inner metallic tubular member extending from a proximal end to a distal end, and an outer metallic tubular member disposed on (e.g., circumferentially surrounding) the inner metallic tubular member. The inner metallic tubular member includes a plurality of interconnected struts defining the inner metallic tubular member. The inner metallic tubular member is moveable between a radially collapsed configuration and a radially expanded configuration. The outer metallic tubular member includes one or more filaments knitted together in a plurality of loops that together form the outer metallic tubular member. The outer metallic tubular member is moveable between a radially collapsed configuration and a radially expanded configuration. The outer metallic tubular member is secured relative to the inner metallic tubular member.

Alternatively or additionally to any of the examples above, in another example, the inner metallic tubular member and/or the outer metallic tubular member comprises nitinol.

Alternatively or additionally to any of the examples above, in another example, the inner metallic tubular member comprises a monolithic tubular member formed of the plurality of interconnected struts.

Alternatively or additionally to any of the examples above, in another example, the outer metallic tubular member comprises an open loop knitted pattern.

Alternatively or additionally to any of the examples above, in another example, the outer metallic tubular member comprises a twisted loop knitted pattern.

Alternatively or additionally to any of the examples above, in another example, an end region of the inner metallic tubular member extends longitudinally beyond the outer metallic tubular member.

Alternatively or additionally to any of the examples above, in another example, at least one of the inner metallic tubular member and the outer metallic tubular member include one or more features that provide a mechanical connection between the inner metallic tubular member and the outer metallic tubular member.

Alternatively or additionally to any of the examples above, in another example, the outer metallic tubular member is secured relative to the inner metallic tubular member via a binder.

Alternatively or additionally to any of the examples above, in another example, the outer metallic tubular member is sutured to the inner metallic tubular member.

Alternatively or additionally to any of the examples above, in another example, the outer metallic tubular member has an outer diameter in an equilibrium configuration of the outer metallic tubular member, and the inner metallic tubular member has an inner diameter in an equilibrium configuration of the inner metallic tubular member that is less than the outer diameter of the outer metallic tubular member in its equilibrium configuration such that the inner metallic tubular member exerts a radially outward force on the outer metallic tubular member in the radially expanded configuration.

Alternatively or additionally to any of the examples above, in another example, the stent includes a drug coating disposed on at least one of the inner metallic tubular member and the outer metallic tubular member.

Another example is a medical stent including an inner tubular member extending from a proximal end to a distal end and a knitted layer circumferentially surrounding an outer surface of the inner tubular member. The inner tubular member is moveable between a radially collapsed configuration and a radially expanded configuration. The inner tubular member is formed of a plurality of interconnected struts defining interstitial spaces therebetween. The knitted layer is formed of one or more interwoven filaments defining interstitial spaces therebetween. The interstitial spaces of the knitted layer are smaller than the interstitial spaces of the inner tubular member.

Alternatively or additionally to any of the examples above, in another example, the inner tubular member comprises a laser cut stent.

Alternatively or additionally to any of the examples above, in another example, the knitted layer comprises an open loop pattern or a twisted loop pattern.

Alternatively or additionally to any of the examples above, in another example, the outer tubular member applies a radially outward force on the knitted layer in the radially expanded configuration.

Alternatively or additionally to any of the examples above, in another example, the knitted layer is mechanically coupled with the inner tubular member.

Alternatively or additionally to any of the examples above, in another example, the stent includes an elutable drug coating on the medical stent.

Alternatively or additionally to any of the examples above, in another example, an end region of the inner tubular member extends longitudinally beyond the knitted layer.

Another example is a medical stent radially expandable from a radially collapsed configuration to a radially expanded configuration. The stent includes an inner tubular member extending from a proximal end to a distal end and an outer tubular member circumferentially surrounding the inner tubular member. The inner tubular member includes a plurality of interconnected struts defining the inner tubular member. The outer tubular member includes one or more filaments knitted together in a plurality of loops that together form the outer tubular member. The outer tubular member has an outer diameter in an equilibrium configuration of the outer tubular member, and the inner tubular member has an inner diameter in an equilibrium configuration of the inner tubular member that is less than the outer diameter of the outer tubular member in its equilibrium configuration such that the inner tubular member exerts a radially outward force on the outer tubular member in the radially expanded configuration.

Alternatively or additionally to any of the examples above, in another example, an end region of the inner tubular member extends longitudinally beyond the outer tubular member.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an example medical stent that includes an inner tubular member and an outer tubular member;

FIG. 2 is a schematic view of an example inner tubular member that forms part of the example medical stent of FIG. 1;

FIG. 3 is a schematic view of an example outer tubular member that may form part of the example medical stent of FIG. 1;

FIG. 4 is an enlarged view of a portion of the example outer tubular member of FIG. 3, showing an open loop knitting pattern;

FIG. 5 is a schematic view of an example outer tubular member that may form part of the example medical stent of FIG. 1;

FIG. 6 is an enlarged view of a portion of the example outer tubular member of FIG. 5, showing a twisted loop knitting pattern;

FIG. 7 is an enlarged view showing a portion of the outer tubular member extending across the inner tubular member;

FIG. 8 is a schematic view of an example inner tubular member, showing an example mechanical attachment feature for securing an outer tubular member thereto;

FIG. 9 is a schematic view of an example inner tubular member, showing an example binding material for securing an outer tubular member thereto;

FIG. 10 is a schematic view of an inner tubular member strut and an outer tubular member loop, showing a suture securing the outer tubular member to the inner tubular member; and

FIG. 11 is a schematic view showing attachment of an example outer tubular member to an example inner tubular member of a stent.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

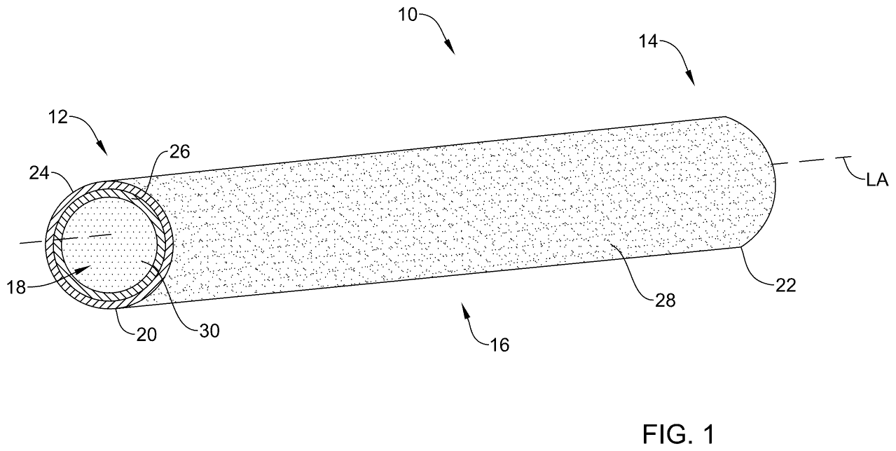

Stents are utilized in a variety of different body lumens, including the vasculature and various parts of the gastrointestinal system, for example. FIG. 1 is a schematic view of an example medical stent 10 that is adapted for use in body lumens in which the medical stent 10 will be subjected to various movement forces. In FIG. 1, the example medical stent 10 is shown as generally tubular, it is contemplated that the medical stent 10 may take any cross-sectional shape desired. The medical stent 10 may be considered as including a first, proximal end region 12, a second, distal end region 14, and an intervening intermediate region 16 extending between the proximal end region 12 and the distal end region 14, recognizing that these designations are arbitrary, and depend on the orientation in which the medical stent 10 will ultimately be implanted within a body lumen. The medical stent 10 may be considered as having a constant diameter throughout, including through the proximal end region 12, the distal end region 14 and the intervening intermediate region 16. In some instances, the medical stent 10 may be considered as having a constant diameter when in a relaxed state. The medical stent 10 may include a lumen 18 that extends from a proximal end 20 of the medical stent 10 to a distal end 22 of the medical stent 10 to allow for the passage of food, fluids, etc. The medical stent 10 may be considered as having a longitudinal axis LA. The proximal end region 12 may extend to the proximal end 20 of the medical stent 10 and the distal end region 14 may extend to the distal end 22 of the medical stent 10. The medical stent 10 may be radially expandable from a first radially collapsed configuration (not shown) to a second radially expanded configuration, which may correspond to its relaxed or equilibrium state. In some instances, the medical stent 10 may be structured to extend across a stricture and to apply a radially outward pressure to the stricture in a lumen in order to open the lumen and allow for the passage of foods, fluids, air, etc.

In some instances, the medical stent 10 includes an outer tubular member 24 and an inner tubular member 26 that together define the structure of the medical stent 10. The outer tubular member 24 may circumferentially surround and be juxtaposed with the outer surface of the inner tubular member 26. In some instances, the inner tubular member 26 may provide all or substantially all of the radial strength of the medical stent 10 while the outer tubular member 24 may not contribute substantially to the radial strength of the medical stent 10. In other words, in some instances the radial outward expansion force generated by the medical stent 10 as the stent 10 transitions from the radially collapsed configuration to the radially expanded configuration may be substantially entirely provided by the inner tubular member 26, while the outer tubular member 24 does not appreciably contribute to the radially outward expansion force generated by the medical stent 10. In some instances, the outer tubular member 24 has an outer diameter in an equilibrium configuration of the outer tubular member 24, and the inner tubular member 26 has an inner diameter in an equilibrium configuration of the inner tubular member 26 that is less than the outer diameter of the outer tubular member 24 in its equilibrium configuration such that the inner tubular member 26 exerts a radially outward force on the outer tubular member 24 in the radially expanded configuration of the medical stent 10.

In some instances, the inner tubular member 26 may be a stent formed of a plurality of interconnected struts formed as a monolithic structure from a tubular member. For example, the inner tubular member 26 may be a laser-cut stent, which is a stent that is laser-cut from a cylinder, such as a metal tube. The EPIC™ stents made by Boston Scientific, Corporation are examples of laser-cut stents. In other instances, the inner tubular member 26 may be a tubular structure formed of one or more, or a plurality of interwoven wires. In some instances, the inner tubular member 26 may be a braided stent formed of a plurality of braided wires, for example. Some exemplary stents including braided filaments include the WallFlex®, WALLSTENT®, and Polyflex® stents, made and distributed by Boston Scientific, Corporation.

In some instances, the outer tubular member 24 may be adapted to provide additional surface for an elutable drug coating, for example. For example, the outer tubular member 24 may be a woven tubular member having smaller interstitial spaces than the inner tubular member 26. In some instances, the outer tubular member 24 may have more surface area than the inner tubular member 26 for an elutable drug coating to be applied thereto. In some instances, the outer tubular member 24 may be a knitted stent, which is a stent formed by knitting one or more wires together. In some instances, a knitted stent may include an open loop knitting pattern. In some instances, a knitted stent may include a twisted loop knitting pattern. The Ultraflex™ stents made by Boston Scientific, Corporation are an example of a knitted stent. In some instances, the outer tubular member 24 may be of a knotted type, such as the Precision Colonic™ stents made by Boston Scientific, Corporation.

In some instances, the outer tubular member 24 is an outer metallic tubular member. In some instances, the inner tubular member 26 is an inner metallic tubular member. In some instances, the inner tubular member 26 may be considered as being a platform for the outer tubular member 24. In some instances, the inner tubular member 26 may be a nitinol platform for the outer tubular member 24, which may also be formed of nitinol.

In some instances, the combination of the outer tubular member 24 and the inner tubular member 26 may enhance transfer of any elutable drug that is disposed on at least one of the outer tubular member 24 and the inner tubular member 26. In some instances, the knit size of the outer tubular member 24 may be adjusted to provide a desired porosity target. In some instances, the outer tubular member 24 and the inner tubular member 26 may each be heat set in order to set a “remembered” configuration prior to assembly of the medical stent 10. In some instances, the outer tubular member 24 may extend the entire length of the inner tubular member 26, and as such, the outer tubular member 24 may be coterminous with the inner tubular member 26. In some instances, the outer tubular member 24 may only extend along a portion of the length of the inner tubular member 26. In some instances, the outer tubular member 24 may provide a smaller void or pore size (e.g., smaller intestinal spaces between adjacent wires or struts), relative to the inner tubular member 26, and as a result the outer tubular member 24 may provide resistance to diseased emboli or other tissue from protruding through the medical stent 10 into the lumen 18 of the medical stent 10. In some instances, the inner tubular member 26 may have a larger void or pore size that by itself may be too large to prevent tissue from protruding through into the lumen 18 of the medical stent 10. The addition of the outer tubular member 24 permit the wires of the outer tubular member 24 to span across the interstitial spaces of the inner tubular member 26 to effectively reduce the area of the interstitial spaces. Such a configuration may help prevent tissue from protruding through the inner tubular member 26 into the lumen 18. In some instances, the outer tubular member 24 may be considered as providing additional support.

In some instances, an inner and/or outer surface of the medical stent 10 may be entirely, substantially, or partially, covered with a polymeric coating. As an example, a polymeric coating 28 is illustrated as a first dotted pattern over an outer surface of the outer tubular member 24. A polymeric coating 30 is illustrated as a second dotted pattern over an inner surface of the inner tubular member 26. The medical stent 10 may include the polymeric coating 28 but not the polymeric coating 30. The medical stent 10 may include the polymeric coating 30 but not the polymeric coating 28. The medical stent 10 may include the polymeric coating 28 and the polymeric coating 30. The medical stent 10 may not include either of the polymeric coating 28 and the polymeric coating 30, and thus may be considered an uncovered medical stent 10. The polymeric coatings 28 and 30, if included, may help reduce tumor or tissue ingrowth into the lumen 18.

In some instances, the medical stent 10 may be a self-expanding stent (SES), meaning that the medical stent 10 will automatically expand into its expanded configuration once any constraints preventing expansion have been removed. In some instances, the medical stent 10 may not be a self-expanding stent, and thus may rely upon an inflatable balloon or other expandable structure within the lumen 18 in order to cause the medical stent 10 to expand from its collapsed configuration for delivery to its expanded configuration for deployment in a body lumen.

The outer tubular member 24 and the inner tubular member 26 may each be formed from a number of different materials such as, but not limited to, metals, metal alloys, shape memory alloys, polymers, as desired, enabling the medical stent 10 to be expanded into shape when accurately positioned within the body. In some instances, the material may be selected to enable the medical stent 10 to be removed with relative ease as well. For example, the outer tubular member 24 and the inner tubular member 26 may each be formed from alloys such as, but not limited to, nitinol and Elgiloy®. In some instances, the outer tubular member 24 may be formed from one or more nitinol filaments. In some instances, the inner tubular member 26 may be laser-cut from a nitinol cylinder or tube.

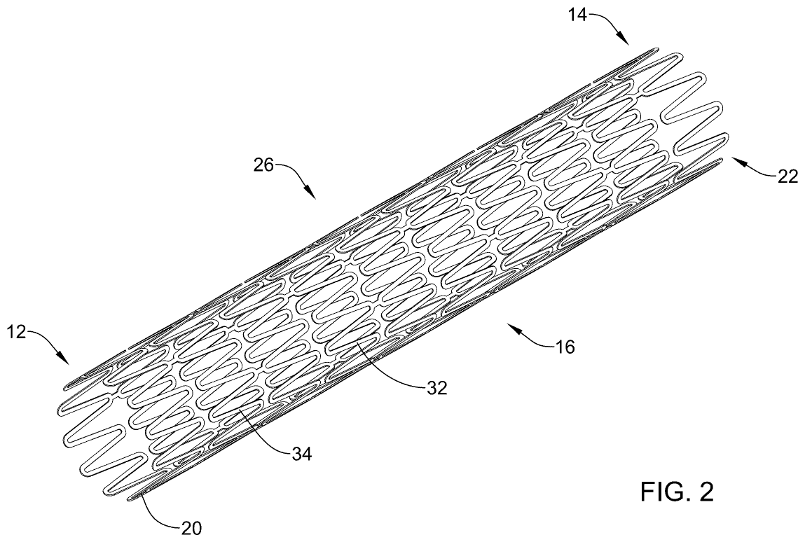

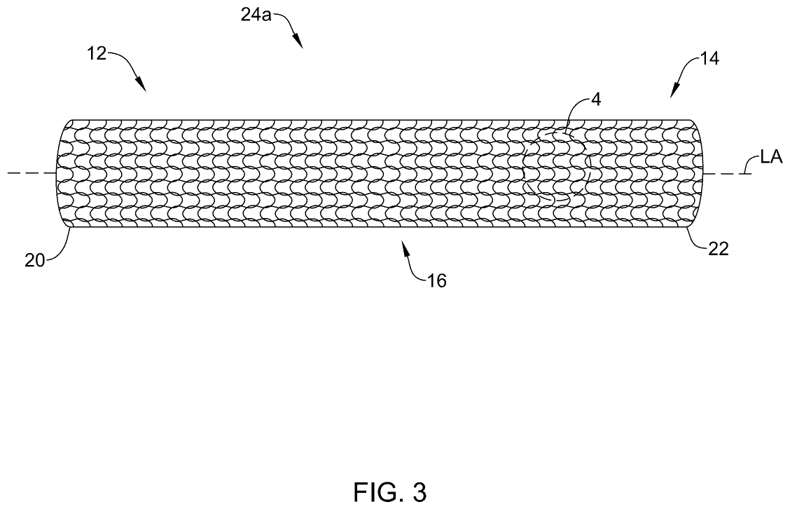

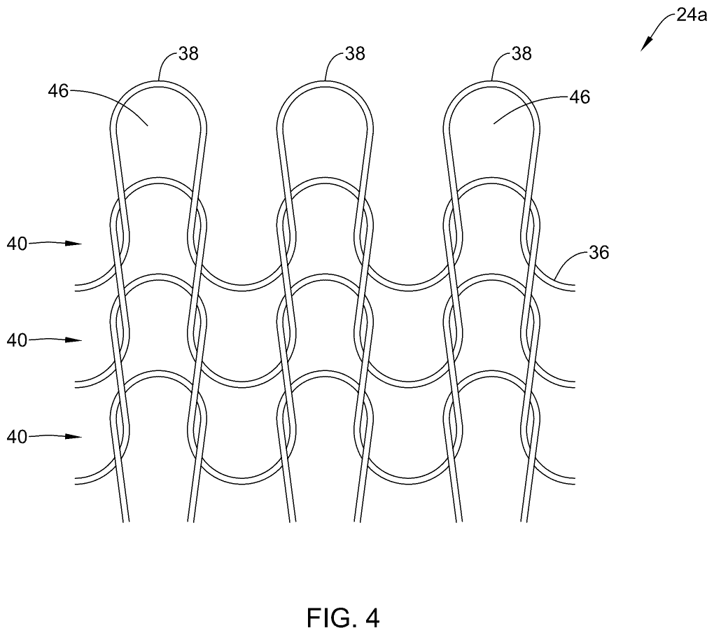

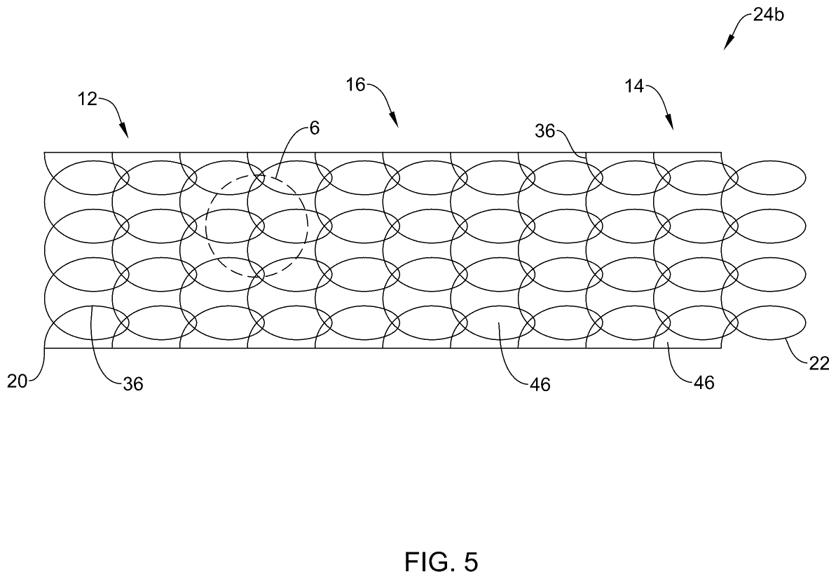

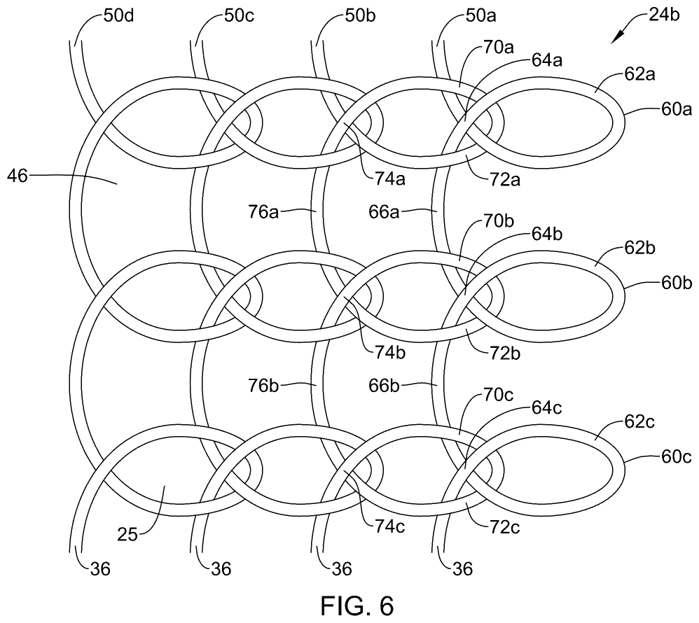

FIG. 2 provides more details regarding the inner tubular member 26, FIGS. 3 and 4 provide more details regarding the outer tubular member 24 formed from one or more filaments or wires knitted into an open loop knitting pattern, and FIGS. 5 and 6 provide more details regarding the outer tubular member 24 formed from one or more filaments or wires knitted into a twisted loop knitting pattern. While FIGS. 3 through 6 provide two examples of possible knitting patterns, it will be appreciated that this is merely illustrative, as any number of different knitting patterns are contemplated and may be utilized in forming the outer tubular member 24.

FIG. 2 is a schematic view of a monolithic tubular member, formed as a laser-cut stent, as an example of the inner tubular member 26. As seen in FIG. 2, the inner tubular member 26 includes an expandable monolithic framework 32. The expandable framework 32 may include a number of interconnected struts 34 to form a monolithic mesh-like structure of the expandable framework 32. The expandable framework extends from a proximal end 20 within a proximal end region 12 to a distal end 22 within a distal end region 14. The struts 34 may be adapted to transition from a radially compressed configuration to a radially expanded configuration, for example. In some instances, the struts 34 may be arranged in a suitable pattern, such as a serpentine configuration, a mesh, a fenestrated pattern, or other arrangement. For example, a number of the struts 34 may form a number of alternating peaks and troughs. The struts 34 may have an inner surface and an outer surface, and a thickness extending between the inner surface and the outer surface. The thickness of the struts 34 may be uniform. In some instances, at least some of the struts 34 may vary. As noted, the struts 34 may be formed by laser-cutting a metal cylinder to remove all of the material that is not the struts 34.

FIG. 3 is a schematic view of a knitted stent 24a that may be considered as being an example of the knitting pattern used to form the outer tubular member 24, and FIG. 4 is an enlarged view of a portion of the knitted stent 24a. In some instances, the outer tubular member 24 may not be a stent such as the knitted stent 24a, but may instead simply be a knitted layer of material that overlays (e.g., wrapped around) the inner tubular member 26. The knitted stent 24a may be considered as having an open loop knitting pattern. The knitted stent 24a extends from a proximal end 20 within a proximal end region 12 to a distal end 22 within a distal end region 14. In some instances, the knitted stent 24a may be produced using an automated weft knitting process that knits a filament 36 into parallel columns 38 of open loops extending longitudinally along the knitted stent 24a and rows 40 of knit stitches extending circumferentially around the knitted stent 24a. The parallel columns 38 run substantially parallel to the longitudinal axis LA of the knitted stent 24a in both the radially expanded, relaxed configuration and an elongated, radially constrained configuration. In some instances, the knitted stent 24a may be formed from a single filament or wire 36, such that each of the rows of knit stiches forming the columns 38 of open loops is formed of only a single filament or wire 36. The knitted stent 24a may be considered as being knitted from a wire or filament 36 that is interwoven with itself, defining interstitial spaces or open cells 46. In some instances, the knitted stent 24a may instead be formed as a spiral knit structure that is formed from a single filament or wire, and instead of distinct rows 40 may have consecutive turns on the spiral. When formed as a spiral knit structure, the knitted stent 24a may have columns 38 that are aligned at a slight angle (e.g., helically arranged) relative to the longitudinal axis LA.

FIG. 5 is a schematic view of a knitted stent 24b that may be considered as being an example of the outer tubular member 24, and FIG. 6 is an enlarged view of a portion of the knitted stent 24b. The knitted stent 24b extends from a proximal end 20 within a proximal end region 12 to a distal end 22 within a distal end region 14. The knitted stent 24b may be considered as being knitted from a wire or filament 36 that is interwoven with itself, defining interstitial spaces or open cells 46. The wire or filament 36 may be a monofilament. In some instances, the filament 36 may be two or more filaments wound, braided, or woven together.

As seen in FIG. 6, the knitted stent 24b may include a plurality of rows 50a, 50b, 50c, 50d (collectively, 50) extending circumferentially about the knitted stent 24b formed from the wire or filament 36. In some instances, the knitted stent 24b may instead be formed as a spiral knit structure from a single filament. The knitted stent 24b may include any number of rows 50 desired, although if formed as a spiral knit structure, the knitted stent 24b may have consecutive turns on the spiral rather than distinct rows. For example, the number of rows 50 may be selected to achieve a desired length of the knitted stent 24b. The uppermost, or first, row 50a may be unsecured and active. In some instances, the first row 50a may include a plurality of closed loops 60a, 60b, 60c (collectively, 60). The closed loops 60 may each include a loop portion 62a, 62b, 62c (collectively, 62) and an overlapping base portion 64a, 64b, 64c (collectively, 64). The overlapping base portion 64a, 64b, 64c is understood as the portion of the closed loops 60 in which one segment of the filament or wire 36 overlaps or crosses over a second segment of the filament or wire 36, with the segment of the filament or wire 36 forming the loop portion 62a, 62b, 62c extending therebetween. Adjacent closed loops 60 may be interconnected by a rung section 66a, 66b (collectively, 66). For example, a first rung section 66a may extend between the base portion 64a of the first loop 60a and the second base portion 64b of the second loop 60b. The next row 50b may be suspended from the loops 60 of the first row 50a. For example, the second, or adjacent row 50b may include a plurality of closed loops 70a, 70b, 70c (collectively, 70) each including a loop portion 72a, 72b, 72c (collectively, 72) and a base portion 74a, 74b, 74c (collectively, 74). Adjacent loops 70 may be interconnected by a rung section 76a, 76b (collectively, 76). As the knitted stent 24b is knitted, the loop portion 60 may extend through the loop 70 of the preceding row 50a. Accordingly, the loop portions 62 of the loops 60 of a first row may wrap around the base portion 64 of the loops 60 of an adjacent, directly preceding row 50.

It is contemplated that a single row 50 may be formed at a time. For example, the rows may be formed in succession with a subsequent row (e.g., row 50b) being formed after the preceding row (e.g., row 50a) has formed a complete rotation. In some instances, when the knitted stent 24b is formed as a spiral knit construction, a row 50 may be considered as being a complete helix of a given number of stitches. While not explicitly shown, the loops 60 of the first row 50a may be wrapped about a section of the filaments 24 free from loops. As described herein, the loops 70 of the second row 50b may be wrapped about the base portion 64 of the loops 60 the preceding row 50a. For example, the filament 36 may be knitted such that it extends from the first rung section 76a, is wrapped about the base portion 64b of the preceding row 50a, crosses back over itself to form base section 74b and continues to the next rung section 76b. It is contemplated that the loop portion 70 may be positioned on a first side of the rungs 66a, 66b and on a second opposite side of the loop portion 62b. In other words, the filament or wire 36 may be wound such that it extends on top of the second rung portion 66b, behind the base portion 64b, and over the first rung portion 66a before crossing over itself to form the base portion 74b of the loop 70b of the second row 50b. The reverse configuration is also contemplated in which the filament or wire 36 may be wound such that it extends behind the second rung portion 66b, over or on top of the base portion 64b, and behind the first rung portion 66a before crossing over itself to form the base portion 74b of the loop 70b of the second row 50b.

In some instances, the outer tubular member 24 and the inner tubular member 26 may be separately formed before being coupled or otherwise assembled together in order to form the medical stent 10. In some instances, the outer tubular member 24 and the inner tubular member 26 may be dimensioned such that the outer tubular member 24 and the inner tubular member 26 are held together via a frictional fit between the outer tubular member 24 and the inner tubular member 26. For example, the outer tubular member 24 may have an inner diameter (when expanded) that is slightly smaller than an outer diameter of the inner tubular member 26 (when expanded). The outer tubular member 24 may be stretched over the inner tubular member 26 and then allowed to relax in order to secure the outer tubular member 24 to the inner tubular member 26. Alternatively, the inner tubular member 26 may be radially compressed, inserted into the outer tubular member 24 in the radially compressed state, and thereafter allowed to radially expand within the outer tubular member 24. It will be appreciated that the difference between the inner diameter of the outer tubular member 24 and the outer diameter of the inner tubular member 26 may be small enough so that the outer tubular member 24 does not materially impact the ability of the inner tubular member 26 to radially expand to its expanded configuration.

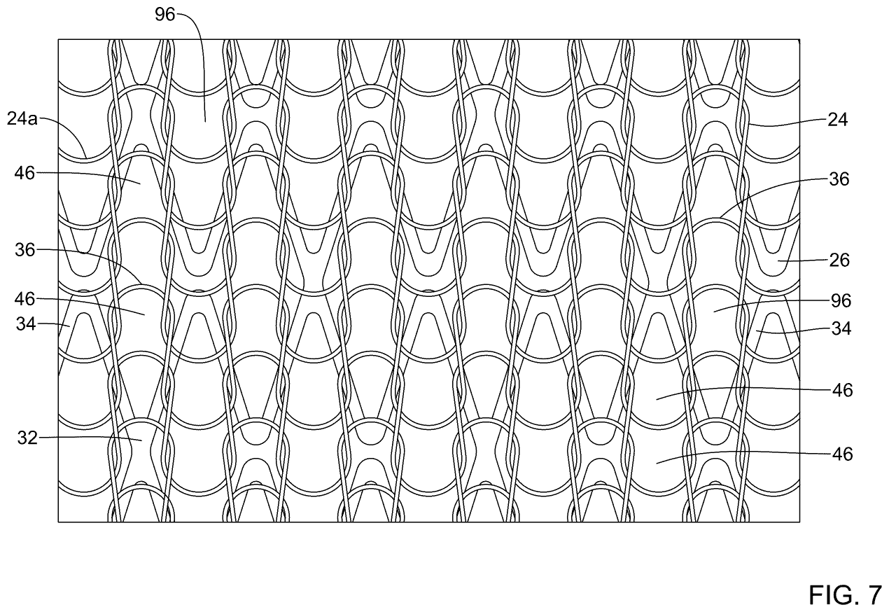

FIG. 7 is an enlarged view showing a portion of the outer tubular member 24 disposed on and extending across the inner tubular member 26. The outer tubular member 24 may circumferentially surround the inner tubular member 26. In some instances, the outer tubular member 24 may extend across or over (e.g., circumferentially surround) the inner tubular member 26 while the filament 36 of the outer tubular member 24 passes under (i.e., radially inward of) some struts 34 of the inner tubular member 26 and passes over (i.e., radially outward of) some struts 34 of the inner tubular member 26 to interlock, attach or otherwise join the outer tubular member 24 to the inner tubular member 26. In such an instance, the filament 36 of the outer tubular member 24 may be described as being interwoven into and out of the inner tubular member 26 through the interstitial spaces or open cells of the inner tubular member 26. In other instances, the entirety of the outer tubular member 24 may be located radially outward of the inner tubular member 26 and be joined thereto by other means, such as by the structures described herein.

As shown in FIG. 7, the outer tubular member 24 is shown having the open loop pattern of the knitted stent 24a shown in FIG. 4, however, in other instances, the outer tubular member 24 may have the twisted loop pattern of the knitted stent 24b of FIG. 6, or another configuration. As discussed above, the inner tubular member 26 may be an expandable framework 32 formed of a plurality of interconnected struts 34 defining interstitial spaces or cells 96 therebetween. The outer tubular member 24, forming a knitted layer, is positioned exterior of the outer surface of the inner tubular member 26 such that the outer tubular member 24 (e.g., knitted layer) circumferentially surrounds the outer surface of the inner tubular member 26. The outer tubular member 24 (e.g., knitted layer) is formed of one or more interwoven filaments 36 (such as a single filament 36) defining interstitial spaces or open cells 46 therebetween. The interstitial spaces or open cells 46 of the outer tubular member 24 (e.g., knitted layer) are smaller than the interstitial spaces or open cells 96 of the inner tubular member 26. As such, the filament 36 of the outer tubular member 24 (e.g., knitted layer) extends across the interstitial spaces or open cells 96 of the inner tubular member 26 effectively dividing the interstitial spaces or open cells 96 into multiple, smaller interstitial spaces or open cells. The medical stent 10 may be considered as an uncovered stent having passageways extending through the tubular wall of the medical stent 10 from an exterior of the medical stent 10 into the lumen of the medical stent 10. The passageways may be defined by the interconnected interstitial spaces or open cells 96 and interstitial spaces or open cells 46.

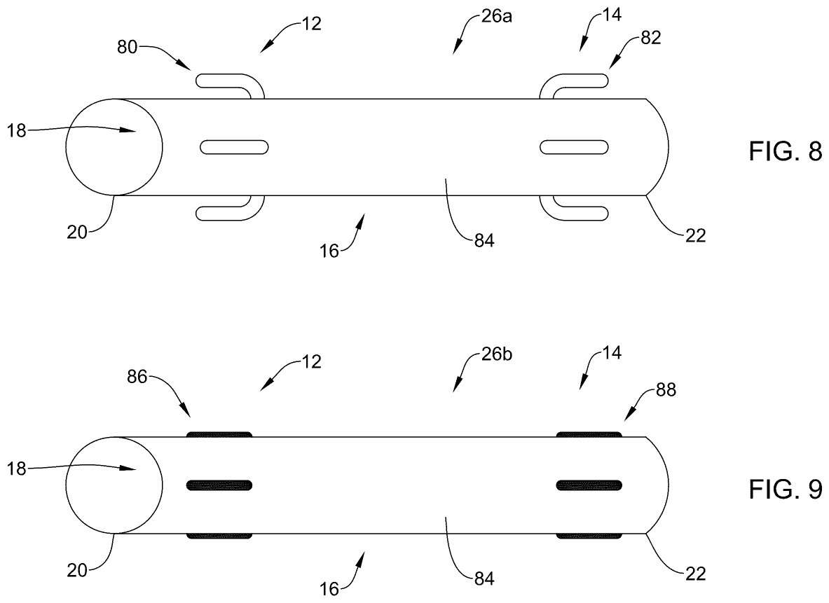

In some instances, the medical stent 10 may include mechanical features that allow the outer tubular member 24 to be coupled to the inner tubular member 26. For example, FIG. 8 is a schematic view of an inner tubular member 26a that may be considered as being an example of the inner tubular member 26. The inner tubular member 26a extends from a proximal end 20 within a proximal end region 12 to a distal end 22 within a distal end region 14, and includes an intervening intermediate region 16. The inner tubular member 26a includes several hooks 80 that are disposed within the proximal end region 12 and several hooks 82 that are disposed within the distal end region 14. The proximal end region 12 may include any number of hooks 80. The distal end region 14 may include any number of hooks 82. The hooks 80 and 82 may be integrally formed as a monolithic part of the inner tubular member 26a when the inner tubular member 26a is laser-cut, for example. In other instances, the hooks 80 and 82 may be separately formed and then welded onto the inner tubular member 26a, for example. In some instances, the hooks 80 within the proximal end region 12 may extend outwardly from an outer surface 84 of the inner tubular member 26a and may extend proximally towards the proximal end 20. In some instances, the hooks 82 within the distal end region 14 may extend outwardly from the outer surface 84 of the inner tubular member 26a and may extend distally towards the distal end 22.

In some instances, the outer tubular member 24 may be stretched over the inner tubular member 26a, or the outer tubular member 24 (in an expanded configuration) may be placed over the inner tubular member 26a (in a collapsed configuration). As the inner tubular member 26a and the outer tubular member 24 relax, the hooks 80 and 82 will catch loops formed within the outer tubular member 24, thereby securing the outer tubular member 24 relative to the inner tubular member 26a. In some instances, the inner tubular member 26a may also include additional hooks located elsewhere on the outer surface 84 of the inner tubular member 26a. In some instances, the hooks 80 and 82 may be limited to the proximal end region 12 and the distal end region 14, respectively, in order to allow some play between the inner tubular member 26a and the outer tubular member 24 within the intermediate region 16.

FIG. 9 is a schematic view of an inner tubular member 26b that may be considered as being an example of the inner tubular member 26. The inner tubular member 26b extends from a proximal end 20 within a proximal end region 12 to a distal end 22 within a distal end region 14, and includes an intervening intermediate region 16. The inner tubular member 26b includes several binding materials 86 that are disposed within the proximal end region 12 and several binding materials 88 that are disposed within the distal end region 14. The binding materials 86 and the binding materials 88 may each represent adhesive material that may adhesively secure the outer tubular member 24 to the inner tubular member 26b. Biocompatible polymers such as Corethane® and Bionate® may be used as the binding materials 86 and 88, for example. In some instances, the binding materials 86 and the binding materials 88 may each represent welds that are formed between the inner tubular member 26b and the outer tubular member 24. Binding materials 86, 88 may be other substances that secure or bond the inner tubular member 26b to the outer tubular member 24.

In order to form the medical stent 10, the outer tubular member 24 may be disposed over the inner tubular member 26b, and then the binding materials 86 and 88 may be deposited onto the outer tubular member 24 and/or the inner tubular member 26b. In some instances, the binding materials 86 and 88 may be deposited on the inner tubular member 26b and then the outer tubular member 24 may be disposed over the inner tubular member 26b and attached thereto. In some instances, the inner tubular member 26b may also include additional binding materials located elsewhere on the outer surface 84 of the inner tubular member 26b. In some instances, the binding materials 86 and 88 may be limited to the proximal end region 12 and the distal end region 14, respectively, in order to allow some play between the inner tubular member 26b and the outer tubular member 24 within the intermediate region 16.

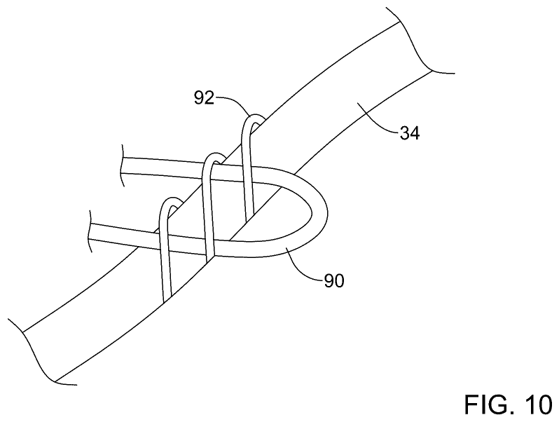

In some instances, the outer tubular member 24 and the inner tubular member 26 may be sutured together. FIG. 10 is a schematic view showing a strut 34 that may be considered as being part of the inner tubular member 26 and a loop 90 that may be considered as part of the outer tubular member 24. The loop 90 of the outer tubular member 24 overlaps the strut 34 and placed against an outer surface of the strut 34 of the inner tubular member 26, and the loop 90 (and hence the outer tubular member 24) is held in position relative to the strut 34 (and hence the inner tubular member 26) via a suture 92 that wraps around both the strut 34 and the loop 90. The suture 92 may represent a suture thread, for example. In some instances, the suture 92 may represent a wire that is wrapped around the strut 34 and the loop 90. In some instances, there may be several sutures 92 used within the proximal end region 12 and several sutures 92 used within the distal end region 14, for example.

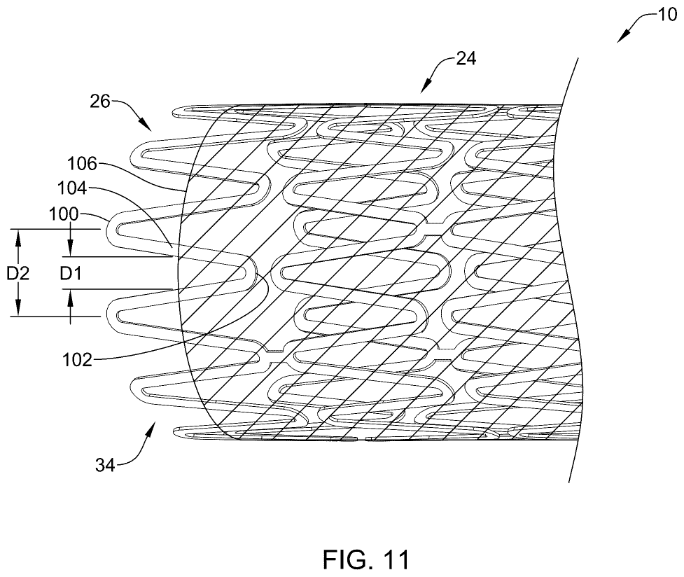

FIG. 11 is a schematic view showing attachment of the outer tubular member 24 to the inner tubular member 26. In some instances, as shown, the inner tubular member 26 may include a number of struts 34 forming undulating circumferential rings. As can be seen, the struts 34 form an undulating pattern, with the struts 34 extending from peaks 100, arranged toward a first end of the inner tubular member 26, to valleys 102, arranged toward a second, opposite end of the inner tubular member 26, with an intermediate extent 104 of the struts 34 disposed between the peaks 100 and the valleys 102. The outer tubular member 24 is disposed circumferentially around the inner tubular member 26 with an end region of the inner tubular member 26 extending beyond the longitudinal extent 106 of the outer tubular member 24. It will be appreciated that the longitudinal extent 106 may represent a distalmost end of the outer tubular member 24, and the peaks 100 of the inner tubular member 24 extending beyond the longitudinal extent 106 of the outer tubular member 24 may represent a distalmost end of the inner tubular member 24. Similarly, the longitudinal extent 106 may represent a proximalmost end of the outer tubular member 24, and the peaks 100 of the inner tubular member 24 extending beyond the longitudinal extent 106 of the outer tubular member 24 may represent a proximalmost end of the inner tubular member 24. It is contemplated that the arrangement of outer and inner tubular members 24, 26 (with peaks 100 of the struts 34 of the inner tubular member 26 extending longitudinally beyond the longitudinal extent 106 of the outer tubular member 24), shown in FIG. 11, may be present at the first, proximal end of the medical stent 10 and/or the second, distal end of the medical stent 10.

As shown in FIG. 11, the longitudinal extent 106 of the outer tubular member 24 may not extend all the way to the longitudinal extent of the inner tubular member 26 such that a portion of the length of the struts 34 in the terminal circumferential row of struts 34 of the inner tubular member 26 extends beyond the outer tubular member 24. For instance, the longitudinal extent (or circumferential edge) of the outer tubular member 24 may instead be aligned or substantially aligned with the intermediate extent 104 of the terminal row of struts 34 of the inner tubular member 26. It will be appreciated that at the intermediate extent 104 of the terminal row of struts 34 of the inner tubular member 26, a distance D₁ between adjoining undulations of struts 34 is less than a distance D₂ measured at the peaks 100 of the undulations of struts 34. In other words, the circumferential distance D₁ that longitudinal extent or edge of the outer tubular member 24 spans across adjacent struts 34 of the terminal circumferential row of struts 34 of the inner tubular member 26 is less than the circumferential distance D₂ between adjacent peaks 100 of the undulating struts 34 of the terminal circumferential row of struts 34 of the inner tubular member 26. By reducing this distance, the relative distance of unsupported outer tubular member 24 (distance between attachment points) is reduced, and thus the outer tubular member 24 is better constrained against intruding inwardly. The outer tubular member 24 intruding inwardly can negatively impact flow through the medical stent 10.

The materials that can be used for the various components of the medical stent(s), and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical stent, comprising:
an inner metallic tubular member extending from a proximal end to a distal end, the inner metallic tubular member comprising a plurality of interconnected struts defining the inner metallic tubular member, the inner metallic tubular member moveable between a radially collapsed configuration and a radially expanded configuration;
an outer metallic tubular member disposed on the inner metallic tubular member, the outer metallic tubular member comprising one or more filaments knitted together in a plurality of loops that together form the outer metallic tubular member, the outer metallic tubular member moveable between a radially collapsed configuration and a radially expanded configuration;
wherein the outer metallic tubular member is secured relative to the inner metallic tubular member.

2. The medical stent of claim 1, wherein the inner metallic tubular member and/or the outer metallic tubular member comprises nitinol.

3. The medical stent of claim 1, wherein the inner metallic tubular member comprises a monolithic tubular member formed of the plurality of interconnected struts.

4. The medical stent of claim 1, wherein the outer metallic tubular member comprises an open loop knitted pattern.

5. The medical stent of claim 1, wherein the outer metallic tubular member comprises a twisted loop knitted pattern.

6. The medical stent of claim 1, wherein an end region of the inner metallic tubular member extends longitudinally beyond the outer metallic tubular member.

7. The medical stent of claim 1, wherein at least one of the inner metallic tubular member and the outer metallic tubular member include one or more features that provide a mechanical connection between the inner metallic tubular member and the outer metallic tubular member.

8. The medical stent of claim 1, wherein the outer metallic tubular member is secured relative to the inner metallic tubular member via a binder.

9. The medical stent of claim 1, wherein the outer metallic tubular member is sutured to the inner metallic tubular member.

10. The medical stent of claim 1, wherein the outer metallic tubular member has an outer diameter in an equilibrium configuration of the outer metallic tubular member, and the inner metallic tubular member has an inner diameter in an equilibrium configuration of the inner metallic tubular member that is less than the outer diameter of the outer metallic tubular member in its equilibrium configuration such that the inner metallic tubular member exerts a radially outward force on the outer metallic tubular member in the radially expanded configuration.

11. The medical stent of claim 1, further comprising a drug coating disposed on at least one of the inner metallic tubular member and the outer metallic tubular member.

12. A medical stent, comprising:
an inner tubular member extending from a proximal end to a distal end, the inner tubular member moveable between a radially collapsed configuration and a radially expanded configuration, the inner tubular member formed of a plurality of interconnected struts defining interstitial spaces therebetween; and
a knitted layer circumferentially surrounding an outer surface of the inner tubular member, the knitted layer formed of one or more interwoven filaments defining interstitial spaces therebetween;
wherein the interstitial spaces of the knitted layer are smaller than the interstitial spaces of the inner tubular member.

13. The medical stent of claim 12, wherein the inner tubular member comprises a laser cut stent.

14. The medical stent of claim 12, wherein the knitted layer comprises an open loop pattern or a twisted loop pattern.

15. The medical stent of claim 12, wherein the outer tubular member applies a radially outward force on the knitted layer in the radially expanded configuration.

16. The medical stent of claim 12, wherein the knitted layer is mechanically coupled with the inner tubular member.

17. The medical stent of claim 12, further comprising an elutable drug coating on the medical stent.

18. The medical stent of claim 12, wherein an end region of the inner tubular member extends longitudinally beyond the knitted layer.

19. A medical stent radially expandable from a radially collapsed configuration to a radially expanded configuration, the stent comprising:
an inner tubular member extending from a proximal end to a distal end, the inner tubular member comprising a plurality of interconnected struts defining the inner tubular member;
an outer tubular member circumferentially surrounding the inner tubular member, the outer tubular member comprising one or more filaments knitted together in a plurality of loops that together form the outer tubular member;
wherein the outer tubular member has an outer diameter in an equilibrium configuration of the outer tubular member, and the inner tubular member has an inner diameter in an equilibrium configuration of the inner tubular member that is less than the outer diameter of the outer tubular member in its equilibrium configuration such that the inner tubular member exerts a radially outward force on the outer tubular member in the radially expanded configuration.

20. The medical stent of claim 19, wherein an end region of the inner tubular member extends longitudinally beyond the outer tubular member.
